(19) United States
(12) Patent Application Publication
(10) Pub. No.: US 2025/0282946 A1
(43) Pub. Date: Sep. 11, 2025

(54) PLASTIC MOLDED ARTICLES FOR USE AS VAPOR/SUSPENSION DELIVERY DEVICES

(71) Applicant: Eastman Chemical (China) Co., Ltd., Shanghai (CN)

(72) Inventors: Na Rong An, Shanghai (CN); Tian Yi Wu, Shanghai (CN); Brandon Robert Williamson, Johnson City, TN (US)

(73) Assignee: Eastman Chemical (China) Co., Ltd., Kingsport, TN (US)

(21) Appl. No.: 18/862,688

(22) PCT Filed: May 24, 2022
(86) PCT No.: PCT/CN2022/094677

§ 371 Date: Nov. 4, 2024

(51) Int. Cl. C08L 67/06; C08L 69/00

(52) U.S. Cl. CPC C08L 67/06; C08L 69/00

(57) ABSTRACT

An article is provided that comprises a molded component configured to receive a vapor delivery chemical containing composition (“VDC”), the molded component being formed from a copolyester composition comprising a polymeric component that comprises a blend of different polymers, wherein the blend comprises a first copolyester and a second polymer, wherein the first copolyester comprises 1,4-cyclohexanedimethanol and optionally ethylene glycol residues, and the second polymer is polycarbonate (PC).

### FIELD OF THE INVENTION

This invention belongs to the field of polymer-based resins useful for forming articles or components of articles intended for contact with aggressive chemicals in vapor delivery applications. Plastic articles or components for such articles made using these compositions, such as vaporizers, nebulizers, humidifiers, air fresheners, or hand-held vapor delivery devices or components thereof are also provided.

### BACKGROUND OF THE INVENTION

Plastics are a preferred material for making small devices that can be used to deliver a vapor or suspension of a chemical composition based on the relative efficiency of molding parts and articles of various shapes and designs. For example, devices used to deliver/produce a vapor or suspension, such as vaporizers, nebulizers, humidifiers, air fresheners, or hand-held vapor delivery devices, are often manufactured by molding plastic parts that form an assembly to produce the device.

When plastics are used in applications where contact with chemicals will occur, there is the potential for cracking, crazing, softening, etc. of the plastic induced by the chemical environment. Especially aggressive classes of chemicals can include solvents, such as alcohols, glycols, and polyols, and compositions that contain significant levels of such solvents, e.g., E-cig liquid formulations.

Many plastics are adversely affected by these chemicals. Thus, there is a need for plastic materials that have resistance to such chemicals, are easily formed into articles, and maintain acceptable physical properties. Vapor delivery devices can include designs or components with thin walls or could benefit from reduced wall thickness. Further, such device can contain a heating source to produce a vapor. However, such design constraints or attributes would make it more difficult to produce articles/devices that maintain dimensional stability due to exposure to higher temperatures and/or reduced wall thickness.

It would be beneficial to be able to provide polymer-based resins that can be melt processed and articles made from such compositions that do not have such drawbacks. It would also be beneficial if such resins could be provided via technology that reduces plastic waste, e.g., technology that includes mechanical or chemical recycling of plastic waste.

### SUMMARY OF THE INVENTION

Articles molded from certain copolyester plastics have good to exceptional resistance to certain aggressive chemicals (e.g., certain solvents and oils) that may be found in chemical vapor or suspension formulations that are used in devices to deliver such formulations (“vapor delivery chemicals”), while also maintaining sufficient physical properties required for the intended use of the articles, including article/component designs having reduced wall thickness. In embodiments, such articles are useful as containers and/or other components in vapor delivery devices that will have significant contact with such aggressive chemicals (vapor delivery chemicals) in use. In one aspect, articles configured to receive a vapor delivery chemical containing composition can be made from compositions of copolyesters that can be prepared having good chemical resistance to the vapor delivery chemical containing composition and a heat distortion temperature (HDT) exceeding 85° C., or 90° C., or 95° C., or 100° C., measured per ASTM D648 at 0.45 MPa load.

Shaped articles configured to receive such a composition can be prepared from copolyester plastic materials that have resistance to the aggressive chemicals (contained in the composition) and have physical properties similar to or better than molded articles produced from other typically used oil-based engineering thermoplastics. More specifically, depending on the desired use these shaped articles are produced from a copolyester composition that can retain physical properties better than other plastics after exposure to certain aggressive chemicals.

Shaped articles configured to receive a vapor delivery chemical containing composition and comprising a copolyester composition can be provided, wherein the copolyester composition has a HDT of at least 85° C., or at least 90° C., and has at least one of the following properties chosen from: tensile modulus of greater than 1600 MPa, or greater than 1800 MPa, as measured according to ASTM D638 using a 3.2 mm thick bar that has been subjected to 50% relative humidity for 40 hours at 23° C.; a notched izod impact strength of greater than 50, or greater than 80, or greater than 100 J/m as measured according to ASTM D256 at 23 C using a 3.2 mm thick bar that has been subjected to 50% relative humidity for 40 hours at 23° C.; a tensile stress at yield of at least 30, or at least 40, or at least 50 MPa, measured according to ASTM D638; or a haze value less than 20, or less than 10, measured according to ASTM D1003 using a 3.2 mm plaque after injection molding at a barrel set point of 250 to 270° C. and a mold temperature of 50° C. In embodiments, the copolyester composition has at least 2, or at least 3 of the listed properties.

The shaped articles or components thereof can be chosen from injection molded articles, extrusion molded articles, rotational molded articles, compression molded articles, blow molded articles, injection blow molded articles, injection stretch blow molded articles, extrusion blow molded articles, sheet or film extrusion articles, profile extrusion articles, gas assist molding articles, structural foam molded articles, or thermoformed articles. The shaped articles can be opaque articles, transparent articles, see-through articles, thin-walled articles, technical articles (e.g., articles having a complex design), articles having high design specifications, intricate design articles, containers for holding a vapor delivery chemical containing composition, or other shaped articles configured to receive (or contact) a vapor delivery chemical containing composition. Technical articles, i.e., articles having high design specifications, and intricate design articles can be articles that include electrical/electronic components, perfume or cosmetic containers, vapor delivery devices, or components thereof.

In one aspect, articles configured to receive a vapor delivery chemical containing composition (“VDC”) are provided that comprise a copolyester composition, wherein the copolyester composition comprises a polymeric component that comprises a copolyester having terephthalic acid residues and cyclohexanedimethanol residues, and optionally residues of at least one other diol (“PCTG Polyester”). In embodiments, the copolyester composition can include at least one PCTG Polyester which comprises:
(a) a dicarboxylic acid component comprising:
   i) 50 to 100 mole % of terephthalic acid residues;
(b) a glycol component comprising:
   i) 0 to 50 mole % of ethylene glycol residues; and
   ii) 50 to 100 mole % of 1,4-cyclohexanedimethanol residues, wherein the total mole % of the dicarboxylic acid component is 100 mole %, and the total mole % of the glycol component is 100 mole %; and have an inherent viscosity of 0.50 to 1.2 dL/g as determined in 60/40 (wt/wt) phenol/tetrachloroethane at a concentration of 0.5 g/100 ml at 25° C.; and wherein the polyester has a HDT of 65° C. to 100° C., measured per ASTM D648 at 0.45 MPa load.

More specific copolyester compositions can include at least one PCTG Polyester which comprises:
(a) a dicarboxylic acid component comprising:
   i) 70 to 100 mole % of terephthalic acid residues;
(b) a glycol component comprising:
   i) 5 to 50 mole % of ethylene glycol residues; and
   ii) 50 to 95 mole % of 1,4-cyclohexanedimethanol residues, wherein the total mole % of the dicarboxylic acid component is 100 mole %, and the total mole % of the glycol component is 100 mole %; and wherein the inherent viscosity is 0.60 to 1.0 dL/g as determined in 60/40 (wt/wt) phenol/tetrachloroethane at a concentration of 0.5 g/100 ml at 25° C.; and wherein the polyester has a HDT of 70° C. to 90° C., measured per ASTM D648 at 0.45 MPa load.

For certain compositions, the dicarboxylic acid component can comprise: 75 to 100 mole % of terephthalic acid (TPA/DMT) residues; and 0 to 25 mole % of isophthalic acid (IPA) residues. In embodiments, the dicarboxylic acid component comprises residues as follows: greater than 75 to 100 mole % TPA and 0 to less than 25 mole % IPA; 80 to 100 mole % TPA and 0 to 20 mole % IPA; 85 to 100 mole % TPA and 0 to 15 mole % IPA; 90 to 100 mole % TPA and 0 to 10 mole % IPA; 95 to 100 mole % TPA and 0 to 5 mole % IPA; 75 to 99 mole % TPA and 1 to 25 mole % IPA; 75 to 98 mole % TPA and 2 to 25 mole % IPA; 75 to 97 mole % TPA and 3 to 25 mole % IPA; 75 to 96 mole % TPA and 4 to 25 mole % IPA; 75 to 95 mole % TPA and 5 to 25 mole % IPA; 80 to 99 mole % TPA and 1 to 20 mole % IPA; 80 to 98 mole % TPA and 2 to 20 mole % IPA; 80 to 97 mole % TPA and 3 to 20 mole % IPA; 80 to 96 mole % TPA and 4 to 20 mole % IPA; 80 to 95 mole % TPA and 5 to 20 mole % IPA; 90 to 99 mole % TPA and 1 to 10 mole % IPA; 90 to 98 mole % TPA and 2 to 10 mole % IPA; 90 to 97 mole % TPA and 3 to 10 mole % IPA; 90 to 96 mole % TPA and 4 to 10 mole % IPA; or 90 to 95 mole % TPA and 5 to 10 mole % IPA.

In embodiments, the dicarboxylic acid component comprises residues as follows: greater than 95 to 100 mole % TPA and 0 to less than 25 mole % IPA; 96 to 100 mole % TPA and 0 to 4 mole % IPA; 96.5 to 100 mole % TPA and 0 to 3.5 mole % IPA; 97 to 100 mole % TPA and 0 to 3 mole % IPA; 98 to 100 mole % TPA and 0 to 2 mole % IPA; 98.5 to 100 mole % TPA and 0 to 1.5 mole % IPA; 95 to 98.5 mole % TPA and 1.5 to 5 mole % IPA; greater than 95 to 98.5 mole % TPA and 1.5 to less than 5 mole % IPA; 96 to 98.5 mole % TPA and 1.5 to 4 mole % IPA; 96.5 to 98.5 mole % TPA and 1.5 to 3.5 mole % IPA; 97 to 98.5 mole % TPA and 1.5 to 3 mole % IPA; 97.5 to 98.5 mole % TPA and 1.5 to 2.5 mole % IPA; 95 to 98 mole % TPA and 2 to 5 mole % IPA; greater than 95 to 98 mole % TPA and 2 to less than 5 mole % IPA; 96 to 98 mole % TPA and 2 to 4 mole % IPA; 96.5 to 98 mole % TPA and 2 to 3.5 mole % IPA; or 97 to 98 mole % TPA and 2 to 3 mole % IPA. For certain compositions, the dicarboxylic acid component has 100 mole % of terephthalic acid (TPA) residues.

For certain compositions, the glycol component comprises: 20 to 50 mole % of ethylene glycol (EG) residues; and 50 to 80 mole % of 1,4-cyclohexanedimethanol (CHDM) residues. In embodiments, the glycol component comprises: 25 to 49 mole % of ethylene glycol (EG) residues; and 51 to 75 mole % of 1,4-cyclohexanedimethanol (CHDM) residues. For certain compositions, the glycol component comprises: 33 to 43 mole % of ethylene glycol (EG) residues; and 57 to 67 mole % of 1,4-cyclohexanedimethanol (CHDM) residues.

In addition to the diols set forth above, in certain embodiments the PCTG Polyester may also be made from 1,3-propanediol, 1,4-butanediol, or mixtures thereof. It is contemplated that compositions made from 1,3-propanediol, 1,4-butanediol, or mixtures thereof can possess at least one of the HDT ranges described herein, at least one of the inherent viscosity ranges described herein, and/or at least one of the glycol or diacid ranges described herein. In addition or in the alternative, the PCTG Polyester made from 1,3-propanediol or 1,4-butanediol or mixtures thereof may also be made from 1,4-cyclohexanedmethanol in at least one of the following amounts: from 0.1 to 99 mole %; from 0.1 to 90 mole %; from 0.1 to 80 mole %; from 0.1 to 70 mole %; from 0.1 to 60 mole %; from 0.1 to 50 mole %; from 0.1 to 40 mole %; from 0.1 to 35 mole %; from 0.1 to 30 mole %; from 0.1 to 25 mole %; from 0.1 to 20 mole %; from 0.1 to 15 mole %; from 0.1 to 10 mole %; from 0.1 to 5 mole %; from 1 to 99 mole %; from 1 to 90 mole %, from 1 to 80 mole %; from 1 to 70 mole %; from 1 to 60 mole %; from 1 to 50 mole %; from 1 to 40 mole %; from 1 to 35 mole %; from 1 to 30 mole %; from 1 to 25 mole %; from 1 to 20 mole %; from 1 to 15 mole %; from 1 to 10 mole %; from 1 to 5 mole %; from 5 to 99 mole %, from 5 to 90 mole %, from 5 to 80 mole %; 5 to 70 mole %; from 5 to 60 mole %; from 5 to 50 mole %; from 5 to 40 mole %; from 5 to 35 mole %; from 5 to 30 mole %; from 5 to 25 mole %; from 5 to 20 mole %; and from 5 to 15 mole %; from 5 to 10 mole %; from 10 to 99 mole %; from 10 to 90 mole %; from 10 to 80 mole %; from 10 to 70 mole %; from 10 to 60 mole %; from 10 to 50 mole %; from 10 to 40 mole %; from 10 to 35 mole %; from 10 to 30 mole %; from 10 to 25 mole %; from 10 to 20 mole %; from 10 to 15 mole %; from 20 to 99 mole %; from 20 to 90 mole %; from 20 to 80 mole %; from 20 to 70 mole %; from 20 to 60 mole %; from 20 to 50 mole %; from 20 to 40 mole %; from 20 to 35 mole %; from 20 to 30 mole %; and from 20 to 25 mole.

In certain embodiments, the glycol component of the PCTG Polyester can contain 25 mole % or less of one or more modifying glycols which are not ethylene glycol or 1,4-cyclohexanedimethanol; in one embodiment, the PCTG Polyester useful in the invention may contain less than 15 mole % of one or more modifying glycols. In another embodiment, the PCTG Polyester can contain 10 mole % or less of one or more modifying glycols. In another embodiment, the PCTG Polyester can contain 5 mole % or less of one or more modifying glycols. In another embodiment, the PCTG Polyester can contain 3 mole % or less of one or more modifying glycols. In another embodiment, the PCTG Polyester can contain 0 mole % modifying glycols. Certain embodiments can also contain 0.01 or more mole %, such as 0.1 or more mole %, 1 or more mole %, 5 or more mole %, or 10 or more mole % of one or more modifying glycols. Thus, if present, it is contemplated that the amount of one or more modifying glycols can range from any of these preceding endpoint values including, for example, from 0.01 to 15 mole % and from 0.1 to 10 mole %.

In embodiments, modifying glycols useful in the PCTG Polyester refer to diols other than ethylene glycol and 1,4-cyclohexanedimethanol and may contain 2 to 16 carbon atoms. Examples of suitable modifying glycols in certain embodiments include, but are not limited to, 1,2-propanediol, 1,3-propanediol, neopentyl glycol, 1,4-butanediol, 1,5-pentanediol, 1,6-hexanediol, 1,10-decanediol, diethylene glycol, triethylene glycol, resorcinol, isosorbide, 2-methyl-1,3-propanediol, p-xylene glycol or mixtures thereof. In another embodiment, the modifying glycols are 1,3-propanediol and/or 1,4-butanediol. In another embodiment, 1,3-propanediol and 1,4-butanediol are excluded as modifying diols. In another embodiment, 2,2-dimethyl-1,3-propanediol is excluded as a modifying diol.

In embodiments, the PCTG Polyester useful in the polyester compositions can comprise from 0 to 10 mole percent, for example, from 0.01 to 5 mole percent, from 0.01 to 1 mole percent, from 0.05 to 5 mole percent, from 0.05 to 1 mole percent, or from 0.1 to 0.7 mole percent, based the total mole percentages of either the diol or diacid residues; respectively, of one or more residues of a branching monomer, also referred to herein as a branching agent, having 3 or more carboxyl substituents, hydroxyl substituents, or a combination thereof. In certain embodiments, the branching monomer or agent may be added prior to and/or during and/or after the polymerization of the PCTG Polyester.

In another aspect, articles configured to receive a vapor delivery chemical containing composition (“VDC”) are provided that comprise a copolyester composition, wherein the copolyester composition comprises a polymeric component that comprises a blend of different polymers. In embodiments, the blend comprises a first copolyester having terephthalic acid residues and cyclohexanedimethanol residues, and optionally residues of at least one other diol (“PCTG Polyester”) and a second polymer that is a polycarbonate. In embodiments, the blend provides a composition with at least one enhanced property compared to a composition where the polymeric component is only the first copolyester.

In another aspect, use of a copolyester composition for making an article configured to receive a vapor delivery chemical containing composition (“VDC”), or a component of said article, is provided. In embodiments, the copolyester composition comprises a polymeric component that comprises a blend of different polymers. In embodiments, the blend comprises a first copolyester having terephthalic acid residues and cyclohexanedimethanol residues, and optionally residues of at least one other diol (“PCTG Polyester”) and a polycarbonate. In embodiments, the blend provides a composition with at least one enhanced property compared to a composition where the polymeric component is only the first copolyester. In embodiments, use of a copolyester composition (as described herein) for containing and/or transporting a vapor delivery chemical containing composition (“VDC”) is provided. In embodiments, use of a copolyester composition (as described herein) for containing and/or transporting a vapor delivery chemical containing composition (“VDC”) in liquid form, and for delivering said VDC in vapor form, is provided. In embodiments, use of a copolyester composition (as described herein) in connection with (or as an integral part of) a vapor delivery device to provide a contact surface for a vapor delivery chemical containing composition (“VDC”) is provided.

In embodiments, the copolyester composition is amorphous. In other embodiments, the copolyester composition is semi-crystalline. In embodiments, the at least one PCTG Polyester is a reactor grade polyester prepared by a process that includes a transesterification reaction of reaction mixture that includes all the monomers for the intended (monomeric) residues to be included in the copolyester. For example, a copolyester intended to include residues of TPA, CHDM and EG is prepared by a transesterification reaction that includes each of these monomers. In an embodiment, the reactor grade PCTG Polyester is amorphous.

In embodiments, a system for vapor delivery of a vapor delivery chemical containing composition is provided that comprises a shaped article configured to receive a vapor delivery chemical containing composition and a vapor delivery chemical containing composition, wherein the shaped article comprises one or more surfaces in contact with the vapor delivery chemical containing composition and/or configured to contact the vapor delivery chemical containing composition when the system is used for its intended purpose, and wherein the one or more surfaces are formed from a copolyester composition (as described herein). In embodiments, a majority of the surfaces that are in contact with the vapor delivery chemical containing composition and/or configured to contact the vapor delivery chemical containing composition when the system is used for its intended purpose are formed from the copolyester composition.

In embodiments, the vapor delivery chemical containing composition is in the form of a liquid and/or a vapor. In embodiments, the system comprises a shaped article that comprises one or more liquid contact surfaces in contact with a liquid vapor delivery chemical containing composition and one or more vapor contact surfaces configured to contact a vapor delivery chemical containing composition in a vapor form when the system is used for its intended purpose. In one embodiment, the one or more liquid contact surfaces and the one or more vapor contact surfaces are in fluid communication and the vapor (of the vapor delivery chemical containing composition) is produced by vaporizing the liquid vapor delivery chemical containing composition. In one embodiment, the system comprises a shaped article that comprises one or more surfaces in contact with both a liquid and a vapor (of the vapor delivery chemical containing composition).

In embodiments, the system comprises a shaped article that comprises one or more liquid contact surfaces in contact with a liquid form of the vapor delivery chemical containing composition for at least 5 minutes. In embodiments, the system comprises a shaped article that comprises one or more vapor contact surfaces in contact with a vapor form of the vapor delivery chemical containing composition repetitively for a total contact time of at least 5 minutes.

In embodiments, the vapor delivery chemical containing composition comprises a vapor delivery chemical that is present in an amount of at least 0.1, or at least 0.2, or at least 0.5, or at least 1 wt %, based on the total weight of the vapor delivery chemical containing composition.

### DETAILED DESCRIPTION

The term “polyester”, as used herein, is intended to include “copolyesters” and is understood to mean a synthetic polymer prepared by the reaction of one or more difunctional carboxylic acids and/or multifunctional carboxylic acids with one or more difunctional hydroxyl compounds and/or multifunctional hydroxyl compounds. Typically, the difunctional carboxylic acid can be a dicarboxylic acid and the difunctional hydroxyl compound can be a dihydric alcohol such as, for example, glycols and diols. The term “glycol” as used in this application includes, but is not limited to, diols, glycols, and/or multifunctional hydroxyl compounds, for example, branching agents. Alternatively, the difunctional carboxylic acid may be a hydroxy carboxylic acid such as, for example, p-hydroxybenzoic acid, and the difunctional hydroxyl compound may be an aromatic nucleus bearing 2 hydroxyl substituents such as, for example, hydroquinone. The term “residue”, as used herein, means any organic structure incorporated into a polymer through a polycondensation and/or an esterification reaction from the corresponding monomer. The term “repeating unit”, as used herein, means an organic structure having a dicarboxylic acid residue and a diol residue bonded through a carbonyloxy group. Thus, for example, the dicarboxylic acid residues may be derived from a dicarboxylic acid monomer or its associated acid halides, esters, salts, anhydrides, or mixtures thereof. As used herein, therefore, the term dicarboxylic acid is intended to include dicarboxylic acids and any derivative of a dicarboxylic acid, including its associated acid halides, esters, half-esters, salts, half-salts, anhydrides, mixed anhydrides, or mixtures thereof, useful in a reaction process with a diol to make polyester. Furthermore, as used in this application, the term “diacid” includes multifunctional acids, for example, branching agents. As used herein, the term “terephthalic acid” is intended to include terephthalic acid itself and residues thereof as well as any derivative of terephthalic acid, including its associated acid halides, esters, half-esters, salts, half-salts, anhydrides, mixed anhydrides, or mixtures thereof or residues thereof useful in a reaction process with a diol to make polyester.

In embodiments, the polyester includes an acid component that comprises terephthalic acid residues. In embodiments, terephthalic acid may be used as the starting material. In other embodiments, dimethyl terephthalate may be used as the starting material. In yet other embodiments, mixtures of terephthalic acid and dimethyl terephthalate may be used as the starting material and/or as an intermediate material. In embodiments, at least a portion of the terephthalic acid or dimethyl terephthalate used as a starting material has recycle content derived directly or indirectly from recycle waste. In embodiments, the recycle content can be obtained from waste plastic that contains terephthalic acid residues, e.g., recovered monomers obtained through a solvolysis (e.g., methanolysis) process. In embodiments, the terephthalic acid residues present in the polyester (according to any of the embodiments herein) contains at least 50 mole %, or at least 75 mole %, or 100 mole % recycle content. In embodiments, the dicarboxylic acid component of the polyester comprises monomer residues having at least 50 mole % recycle content, or at least 75 mole % recycle content, or 100 mole % recycle content.

In embodiments, the polyester includes a diol component that comprises CHDM and/or EG residues. In embodiments, at least a portion of the CHDM and/or EG used as a starting material has recycle content derived directly or indirectly from recycle waste. In embodiments, the recycle content can be obtained from waste plastic that contains CHDM and/or EG residues, e.g., recovered monomers obtained through a solvolysis (e.g., methanolysis) process. In embodiments, the CHDM and/or EG residues present in the PCTG Polyester (according to any of the embodiments herein) contains at least 50 mole %, or at least 75 mole %, or 100 mole % recycle content. In embodiments, the glycol component of the PCTG Polyester comprises monomer residues having at least 50 mole % recycle content, or at least 75 mole % recycle content, or 100 mole % recycle content.

The polyester (as described herein) can have (or include) a recycle content that is provided by chemical recycling where waste material is broken down into small molecules that are then used to make the polyester, e.g., a waste stream (e.g., containing waste plastic) is gasified to produce syngas and the syngas is then utilized in one or more reaction schemes to produce the polyester.

A recycle content polyester can also be provided that has (or includes) recycle content using a mass balance approach. In a mass balance approach, a recycle content value is determined and then applied or associated with the polyester. A “recycle content value” is a unit of measure representative of a quantity of material having its origin in recycled waste, e.g., recycled plastic. The particular recycle content value can be determined by a mass balance approach or a mass ratio or percentage or any other unit of measure and can be determined according to any system for tracking, allocating, and/or crediting recycle content among various compositions. A recycle content value can be deducted from a recycle content inventory and applied to a product or composition (e.g., the polyester) to attribute recycle content to the product or composition (e.g., the polyester). A recycle content value can come from waste material (e.g., mixed waste plastic) and can be applied to the polyester based on a mass balance approach that takes into account the stoichiometry and efficiencies of the processes used to make the polyester.

The recycled content in the polyester can be at least partially derived from recycled polyester of the same type, providing a circular recycling solution. The circular recycling solution can include determining recycle content value (or credits) for waste polyester of the same type and applying at least a portion of such recycle value or credit to the new polyester (e.g., by a mass balance approach), or can be a closed loop process for providing a recycle polyester where at least a portion of the feedstock utilized in the process/reaction scheme to make the polyester is obtained from the same polyester type. In one aspect, the closed loop process is based on chemical recycling and not mechanical recycling.

In certain aspects, the closed loop can include end of life vapor delivery articles being used as feedstock to provide recycle content to renewed vapor delivery articles containing recycle content polyester compositions (as described herein). A closed loop process is differentiated from an open loop process in that the renewed articles made in an open loop process are different from the end of life articles recycled as a feedstock material. The match between recycled articles and renewed material made in a closed loop system does not have to be compositionally identical, e.g., the recycled articles can have a different polymer formulation but have a similar based polyester with the same types of monomer residues. The process to provide recycle content can be operated as a closed loop process and an open loop process simultaneously.

In various aspects, the polyester composition used to make the articles (as described herein) contains at least 10, or at least 15, or at least 20, or at least 25, or at least 30, or at least 40, or at least 50, or at least 55, or at least 60, or at least 65, or at least 70, or at least 75, or at least 80, or at least 85, or at least 90, or at least 95, or 100 wt % recycle content, by any of the methods (or combinations of methods) for providing recycle content described herein.

The polyesters used in the present invention typically can be prepared from dicarboxylic acids and diols which react in substantially equal proportions and are incorporated into the polyester polymer as their corresponding residues. The polyesters of the present invention, therefore, can contain substantially equal molar proportions of acid residues (100 mole %) and diol (and/or multifunctional hydroxyl compounds) residues (100 mole %) such that the total moles of repeating units is equal to 100 mole %. The mole percentages provided in the present disclosure, therefore, may be based on the total moles of acid residues, the total moles of diol residues, or the total moles of repeating units. For example, a polyester containing 4 mole % isophthalic acid, based on the total acid residues, means the polyester contains 4 mole % isophthalic acid residues out of a total of 100 mole % acid residues. Thus, there are 4 moles of isophthalic acid residues among every 100 moles of acid residues. In another example, a polyester containing 15 mole % ethylene glycol, based on the total diol residues, means the polyester contains 15 mole % ethylene glycol residues out of a total of 100 mole % diol residues. Thus, there are 15 moles of EG residues among every 100 moles of diol residues.

In embodiments, the acid component can comprise furan dicarboxylic acid (FDCA) residues. In embodiments, the polyester comprises an acid component that comprises FDCA, terephthalic acid (TPA) and/or isophthalic acid (IPA) residues and a diol component that comprises CHDM, EG and/or isosorbide residues. In embodiments, the acid component comprises 20 to 100, or 30 to 100, or 40 to 100, or 50 to 100 mole percent of FDCA residues.

In another aspect, the copolyester composition comprises a polymeric component that is a blend of different polymers and the blend comprises a first copolyester having terephthalic acid residues and cyclohexanedimethanol residues, and optionally residues of at least one other diol (“PCTG Polyester”) and a second polymer that is polycarbonate. In embodiments, the blend provides a copolyester composition with at least one enhanced property compared to a composition where the polymeric component is only the first copolyester. In embodiments, the blend provides a copolyester composition with at least one enhanced property compared to a composition where the polymeric component is only the second polymer, where the second polymer is the majority of the polymeric component.

In embodiments, the blend can result in at least one enhanced property chosen from: improved chemical resistance, higher stiffness (e.g., higher tensile modulus), improved heat resistance, improved processability (e.g., better flowability), or lower VOC. In embodiments, the polymeric component comprises a majority of the first copolyester. In other embodiments, the polymeric component comprises a majority of the second polymer.

In embodiments, the second polymer is a polycarbonate (PC). In embodiments, the polycarbonate can be a standard bisphenol-A type polycarbonate. In other embodiments, copolymers and polycarbonates subject to other modifications can be used as well. For example, copolymers of copolyester and polycarbonate are contemplated herein as well. In embodiments, the polycarbonate can have a melt flow rate (MFR), or melt flow index (MFI), in a range from about 15 to about 30 g/10 min, as determined by ASTM D-1238 (300° C./1.2 kg). In other embodiments, the melt flow rate (MFR), or melt flow index (MFI), can be in a range from about 30 (or greater than 30) to about 60 g/10 min, or greater than 30 to 45 g/10 min, as determined by ASTM D-1238 (300° C./1.2 kg). In other embodiments, the melt flow rate (MFR), or melt flow index (MFI), can be in a range from about 60 and about 80 g/10 min, as determined by ASTM D-1238 (300° C./1.2 kg).

Traditional stabilizers, catalysts, impact modifiers, flame retardant agents, reinforcing agents and the like which are well known in the polycarbonate and polyester arts may be used to the extent such additives are of a type and/or in an amount that does not cause the molded article made therefrom to have unacceptable properties, e.g., chemical resistance, optical or strength properties, as described herein. Examples of additives that are contemplated for use include Irgafor® and Irganox® antioxidants, available from BASF and Tinuvin® and Uvinil® light stabilizers, available from BASF.

In embodiments, the copolyester can be a commercially available copolyester resin, such as, for example: Eastar® Copolyester DN011, Eastar® DN004, Eastar® DN001, Spectar, Eastar® MN006, DN004HF, Aspira EB062, and/or Eastar GN078 from Eastman Chemical Company, and/or SKYGREEN® JN200 PCTG from SK Chemicals.

In embodiments, the polycarbonate can be a commercially available polycarbonate resin, such as, for example: Makrolon™ 2458, Makrolon™ 2207, Makrolon™ 2600, Makrolon™ 3101, Makrolon™ 2858, and/or Lexan™ 141. Makrolon resins available from Bayer (Covestro), Lexan resin available from Sabic, and Tarflon resins available from Idemitsu Kosan Company are all examples of polycarbonate trade names.

In embodiments, the PCTG Polymer and PC blend (as described herein) results in a copolyester composition having at least one enhanced property chosen from: improved chemical resistance, higher stiffness (e.g., higher tensile modulus), improved processability (e.g., better flowability), or lower VOC, compared to a copolyester composition where the polymeric component is only the CBDO Polymer. In embodiments, the blend results in two or more of these enhanced properties.

In embodiments, the polymeric composition comprises a blend of PCTG Polymer and PC, wherein the PCTG Polymer is present in an amount of at least 50, or greater than 50, or at least 55, or at least 60, or at least 65 wt %, based on the weight of the copolyester composition. In embodiments, the PC is present in an amount of at 50 wt % or less, or less than 50 wt %, or 45 wt % or less, or 40 wt % or less, or 35 wt % or less, based on the weight of the copolyester composition.

In embodiments, the polymeric composition comprises a blend of PCTG Polymer and PC, wherein the polymers are present in an amount from: 50 to 99 wt % PCTG Polyester and 1 to 50 wt % PC, or more than 50 to 99 wt % PCTG Polyester and 1 to less than 50 wt % PC, or 55 to 99 wt % PCTG Polyester and 1 to 45 wt % PC, or 60 to 99 wt % PCTG Polyester and 1 to 40 wt % PC, or 65 to 99 wt % PCTG Polyester and 1 to 35 wt % PC, or more than 50 to 95 wt % PCTG Polyester and 5 to less than 50 wt % PC, or 55 to 95 wt % PCTG Polyester and 5 to 45 wt % PC, or 60 to 95 wt % PCTG Polyester and 5 to 40 wt % PC, or 65 to 95 wt % PCTG Polyester and 5 to 35 wt % PC, or more than 50 to 90 wt % PCTG Polyester and 10 to less than 50 wt % PC, or 55 to 90 wt % PCTG Polyester and 10 to 45 wt % PC, or 60 to 90 wt % PCTG Polyester and 10 to 40 wt % PC, or 65 to 90 wt % PCTG Polyester and 10 to 35 wt % PC, or more than 50 to 85 wt % PCTG Polyester and 15 to less than 50 wt % PC, or 55 to 85 wt % PCTG Polyester and 15 to 45 wt % PC, or 60 to 85 wt % PCTG Polyester and 15 to 40 wt % PC, or 65 to 85 wt % PCTG Polyester and 15 to 35 wt % PC, or more than 50 to 80 wt % PCTG Polyester and 20 to less than 50 wt % PC, or 55 to 80 wt % PCTG Polyester and 20 to 45 wt % PC, or 60 to 80 wt % PCTG Polyester and 20 to 40 wt % PC, or 65 to 80 wt % PCTG Polyester and 20 to 35 wt % PC, or more than 50 to 75 wt % PCTG Polyester and 25 to less than 50 wt % PC, or 55 to 75 wt % PCTG Polyester and 25 to 45 wt % PC, or 60 to 75 wt % PCTG Polyester and 25 to 40 wt % PC, or 65 to 75 wt % PCTG Polyester and 25 to 35 wt % PC, based on the weight of the copolyester composition.

In embodiments, the polymers are present in an amount from: 60 to 80 wt % PCTG Polyester and 20 to 40 wt % PC, or 62 to 78 wt % PCTG Polyester and 22 to 38 wt % PC, or 64 to 76 wt % PCTG Polyester and 24 to 36 wt % PC, or 66 to 74 wt % PCTG Polyester and 26 to 34 wt % PC, or 67 to 73 wt % PCTG Polyester and 27 to 33 wt % PC, or 68 to 72 wt % PCTG Polyester and 28 to 32 wt % PC, based on the weight of the copolyester composition. In certain embodiments, the above weight percentage can be based just on the combined weight of the PCTG Polymer and PC. In embodiments, the PCTG Polymer is present in an amount greater than the PC. In one embodiment, the PCTG Polymer can be Eastar® Copolyester DN011 (from Eastman) and the PC can be Makrolon® polycarbonate 2458 (from Covestro). In embodiments, the PCTG Polymer and PC blend (as described herein) results in a copolyester composition having at least one enhanced property chosen from: improved chemical resistance, higher stiffness (e.g., higher tensile modulus), improved second thermal processing (e.g., improved cycle and demolding time), compared to a copolyester composition where the polymeric component is only the PCTG Polymer. In embodiments, the blend results in two or more of these enhanced properties.

In embodiments, the HDT of the polyester composition useful in the invention can be at least one of the following ranges: 85 to 140° C.; 85 to 135° C.; 85 to 130° C.; 85 to 125° C.; 85 to 120° C.; 85 to 115° C.; 85 to 110° C.; 85 to 105° C.; 85 to 100° C.; 85 to 95° C.; 90 to 140° C.; 90 to 135° C.; 90 to 130° C.; 90 to 125° C.; 90 to 120° C.; 90 to 115° C.; 90 to 110° C.; 90 to 105° C.; 90 to 100° C.; 95 to 140° C.; 95 to 135° C.; 95 to 130° C.; 95 to 125° C.; 95 to 120° C.; 95 to 115° C.; 95 to 110° C.; 95 to 105° C.; 100 to 140° C.; 100 to 135° C.; 100 to 130° C.; 100 to 125° C.; 100 to 120° C.; 100 to 115° C.; 100 to 110° C.; 105 to 140° C.; 105 to 135° C.; 105 to 130° C.; 105 to 125° C.; 105 to 120° C.; 105 to 115° C.; 110 to 140° C.; 110 to 135° C.; 110 to 130° C.; 110 to 125° C.; 110 to 120° C.; 115 to 140° C.; 115 to 135° C.; 115 to 130° C.; 115 to 125° C.; 120 to 140° C.; 120 to 135° C.; 120 to 130° C.; 125 to 140° C.; 125 to 135° C.; and 130 to 140° C.

For certain applications, the PCTG useful in the blend may exhibit at least one of the following inherent viscosities as determined in 60/40 (wt/wt) phenol/tetrachloroethane at a concentration of 0.5 g/100 ml at 25° C.: 0.60 to 1.2 dL/g; 0.60 to 1.1 dL/g; 0.60 to 1 dL/g; 0.60 to less than 1 dL/g; 0.60 to 0.98 dL/g; 0.60 to 0.95 dL/g; 0.60 to 0.90 dL/g; 0.60 to 0.85 dL/g; 0.60 to 0.80 dL/g; 0.60 to 0.75 dL/g; 0.60 to less than 0.75 dL/g; 0.60 to 0.72 dL/g; 0.60 to 0.70 dL/g; 0.60 to less than 0.70 dL/g; 0.60 to 0.68 dL/g; 0.60 to less than 0.68 dL/g; 0.60 to 0.65 dL/g; 0.65 to 1.2 dL/g; 0.65 to 1.1 dL/g; 0.65 to 1 dL/g; 0.65 to less than 1 dL/g; 0.65 to 0.98 dL/g; 0.65 to 0.95 dL/g; 0.65 to 0.90 dL/g; 0.65 to 0.85 dL/g; 0.65 to 0.80 dL/g; 0.65 to 0.75 dL/g; 0.65 to less than 0.75 dL/g; 0.65 to 0.72 dL/g; 0.65 to 0.70 dL/g; or 0.65 to less than 0.70 dL/g; 0.70 to 1.2 dL/g; 0.70 to 1.1 dL/g; 0.70 to 1 dL/g; 0.70 to less than 1 dL/g; 0.70 to 0.98 dL/g; 0.70 to 0.95 dL/g; 0.70 to 0.90 dL/g; 0.70 to 0.85 dL/g; 0.70 to 0.80 dL/g; 0.70 to 0.75 dL/g; 0.70 to less than 0.75 dL/g; 0.75 to 1.2 dL/g; 0.75 to 1.1 dL/g; 0.75 to 1 dL/g; 0.75 to less than 1 dL/g; 0.75 to 0.98 dL/g; 0.75 to 0.95 dL/g; 0.75 to 0.90 dL/g; 0.75 to 0.85 dL/g; 0.75 to 0.80 dL/g; 0.75 to less than 0.80 dL/g; 0.80 to 1.2 dL/g; 0.80 to 1.1 dL/g; 0.80 to 1 dL/g; 0.80 to less than 1 dL/g; 0.80 to 0.98 dL/g; 0.80 to 0.95 dL/g; 0.80 to 0.90 dL/g; 0.80 to 0.85 dL/g; 0.80 to less than 0.85 dL/g; 0.85 to 1.2 dL/g; 0.85 to 1.1 dL/g; 0.85 to 1 dL/g; 0.85 to less than 1 dL/g; 0.85 to 0.98 dL/g; 0.85 to 0.95 dL/g; 0.85 to 0.90 dL/g; 0.85 to less than 0.90 dL/g; 0.90 to 1.2 dL/g; 0.90 to 1.1 dL/g; 0.90 to 1 dL/g; 0.90 to less than 1 dL/g; 0.90 to 0.98 dL/g; 0.90 to 0.95 dL/g; or 0.90 to less than 0.95 dL/g. It is contemplated that the CBDO Polyester can possess at least one of the inherent viscosity ranges described herein and at least one of the monomer ranges for the compositions described herein unless otherwise stated.

For certain applications, the PC useful in the invention may exhibit at least one of the following melt flow rates: 1 to 40 g/10 minutes, or 2 to 40 g/10 minutes, or 3 to 40 g/10 minutes, or 4 to 40 g/10 minutes, or 5 to 40 g/10 minutes, or 6 to 40 g/10 minutes, or 7 to 40 g/10 minutes, or 8 to 40 g/10 minutes, or 9 to 40 g/10 minutes, or 10 to 40 g/10 minutes, or 15 to 40 g/10 minutes, or 20 to 40 g/10 minutes, or 25 to 40 g/10 minutes, or 30 to 40 g/10 minutes, or 1 to 35 g/10 minutes, or 2 to 35 g/10 minutes, or 3 to 35 g/10 minutes, or 4 to 35 g/10 minutes, or 5 to 35 g/10 minutes, or 6 to 35 g/10 minutes, or 7 to 35 g/10 minutes, or 8 to 35 g/10 minutes, or 9 to 35 g/10 minutes, or 10 to 35 g/10 minutes, or 15 to 35 g/10 minutes, or 20 to 35 g/10 minutes, or 25 to 35 g/10 minutes, or 1 to 30 g/10 minutes, or 2 to 30 g/10 minutes, or 3 to 30 g/10 minutes, or 4 to 30 g/10 minutes, or 5 to 30 g/10 minutes, or 6 to 30 g/10 minutes, or 7 to 30 g/10 minutes, or 8 to 30 g/10 minutes, or 9 to 30 g/10 minutes, or 10 to 30 g/10 minutes, or 15 to 30 g/10 minutes, or 20 to 30 g/10 minutes, or 1 to 25 g/10 minutes, or 2 to 25 g/10 minutes, or 3 to 25 g/10 minutes, or 4 to 25 g/10 minutes, or 5 to 25 g/10 minutes, or 6 to 25 g/10 minutes, or 7 to 25 g/10 minutes, or 8 to 25 g/10 minutes, or 9 to 25 g/10 minutes, or 10 to 25 g/10 minutes, or 15 to 25 g/10 minutes, or 1 to 20 g/10 minutes, or 2 to 20 g/10 minutes, or 3 to 20 g/10 minutes, or 4 to 20 g/10 minutes, or 5 to 20 g/10 minutes, or 6 to 20 g/10 minutes, or 7 to 20 g/10 minutes, or 8 to 20 g/10 minutes, or 9 to 20 g/10 minutes, or 10 to 20 g/10 minutes, wherein melt flow rate (MFR) is measured by CEAST MF 20 according to ASTM-D1238.

It is also contemplated that the polyester composition can possess at least one of the HDT ranges described herein and at least one of the monomer ranges and/or blend ranges for the compositions described herein unless otherwise stated. It is also contemplated that the polyester composition can possess at least one of the HDT ranges described herein, at least one of the inherent viscosity and/or melt flow ranges described herein, and at least one of the monomer or blend ranges for the compositions described herein unless otherwise stated.

In certain embodiments, terephthalic acid, or an ester thereof, such as, for example, dimethyl terephthalate, or a mixture of terephthalic acid and an ester thereof, makes up most or all of the dicarboxylic acid component used to form the polyesters useful in the invention. In certain embodiments, terephthalic acid residues can make up a portion or all of the dicarboxylic acid component used to form the present polyester at a concentration of at least 70 mole %, such as at least 80 mole %, at least 90 mole %, at least 95 mole %, at least 99 mole %, or, in one preferred embodiment (e.g., reactor grade), 100 mole %. In certain embodiments, polyesters with higher amounts of terephthalic acid can be used in order to produce higher impact strength properties. For purposes of this disclosure, the terms “terephthalic acid” and “dimethyl terephthalate” are used interchangeably herein. In one embodiment, dimethyl terephthalate is part or all of the dicarboxylic acid component used to make the polyesters useful in the present invention. In all embodiments, ranges of from 70 to 100 mole %; or 80 to 100 mole %; or 90 to 100 mole %; or 99 to 100 mole %; or 100 mole % terephthalic acid and/or dimethyl terephthalate and/or mixtures thereof may be used.

In certain applications, the polyesters can comprise from 0 to 10 mole percent, for example, from 0.01 to 5 mole percent, from 0.01 to 1 mole percent, from 0.05 to 5 mole percent, from 0.05 to 1 mole percent, or from 0.1 to 0.7 mole percent, or 0.1 to 0.5 mole percent, based the total mole percentages of either the diol or diacid residues; respectively, of one or more residues of a branching monomer, also referred to herein as a branching agent, having 3 or more carboxyl substituents, hydroxyl substituents, or a combination thereof. In certain embodiments, the branching monomer or agent may be added prior to and/or during and/or after the polymerization of the polyester. The polyester(s) useful in the invention can thus be linear or branched. In certain embodiments, the branching monomer or agent may be added prior to and/or during and/or after the polymerization.

Examples of branching monomers include, but are not limited to, multifunctional acids or multifunctional alcohols such as trimellitic acid, trimellitic anhydride, pyromellitic dianhydride, trimethylolpropane, glycerol, pentaerythritol, citric acid, tartaric acid, 3-hydroxyglutaric acid and the like. In one embodiment, the branching monomer residues can comprise 0.1 to 0.7 mole percent of one or more residues chosen from at least one of the following: trimellitic anhydride, pyromellitic dianhydride, glycerol, sorbitol, 1,2,6-hexanetriol, pentaerythritol, trimethylolethane, and/or trimesic acid. The branching monomer may be added to the polyester reaction mixture or blended with the polyester in the form of a concentrate as described, for example, in U.S. Pat. Nos. 5,654,347 and 5,696,176, whose disclosure regarding branching monomers is incorporated herein by reference.

The polyesters can be made by processes known from the literature such as, for example, by processes in homogenous solution, by transesterification processes in the melt, and by two phase interfacial processes. Suitable methods include, but are not limited to, the steps of reacting one or more dicarboxylic acids with one or more glycols at a temperature of 100° C. to 315° C. at a pressure of 0.1 to 760 mm Hg for a time sufficient to form a polyester. See U.S. Pat. No. 3,772,405 for methods of producing polyesters, the disclosure regarding such methods is hereby incorporated herein by reference.

In embodiments, the article made from the copolyester composition can be amorphous. For purposes of this disclosure, amorphous means a crystallinity or less than 1%. In other embodiments, the article made from the copolyester composition, e.g., a PCTG copolyester blend with PC, can be semi-crystalline, e.g., by crystallizing with heat. In embodiments, the article of the invention has a crystallinity of from 1 to 40%, or 1 to 35%, or 1 to 30%, or 5 to 40%, or 5 to 35%, or 5 to 30%, or 10 to 40%, or 10 to 35%, or 10 to 30%.

In addition, the polyester useful in this invention may also contain from 0.01 to 25% by weight or 0.01 to 20% by weight or 0.01 to 15% by weight or 0.01 to 10% by weight or 0.01 to 5% by weight of the total weight of the polyester composition of common additives such as colorants, dyes, mold release agents, reheat additives, flame retardants, plasticizers, stabilizers, including but not limited to, UV stabilizers, thermal stabilizers and/or reaction products thereof, fillers, and impact modifiers. Examples of typical commercially available impact modifiers well known in the art and useful in this invention include, but are not limited to, ethylene/propylene terpolymers; functionalized polyolefins, such as those containing methyl acrylate and/or glycidyl methacrylate; styrene-based block copolymeric impact modifiers; and various acrylic core/shell type impact modifiers. For example, UV additives can be incorporated into articles of manufacture through addition to the bulk, through application of a hard coat, or through coextrusion of a cap layer. Residues of such additives are also contemplated as part of the polyester composition.

In embodiments, the articles (configured to receive vapor deliver chemical containing composition) can include, but are not limited to, injection blow molded articles, injection stretch blow molded articles, extrusion blow molded articles, extrusion stretch blow molded articles, calendered articles, compression molded articles, and solution casted articles. Methods of making the articles of manufacture, include, but are not limited to, extrusion blow molding, extrusion stretch blow molding, injection blow molding, injection stretch blow molding, calendering, compression molding, and solution casting.

In embodiments, the articles (configured to receive a vapor delivery chemical containing composition) can include film(s) and/or sheet(s) comprising the polyester compositions that are formed into the articles. The methods of forming the polyesters into film(s) and/or sheet(s) are well known in the art. Examples of film(s) and/or sheet(s) of the invention including but not limited to extruded film(s) and/or sheet(s), calendered film(s) and/or sheet(s), compression molded film(s) and/or sheet(s), solution casted film(s) and/or sheet(s). Methods of making film and/or sheet include but are not limited to extrusion, calendering, compression molding, and solution casting.

The copolyester composition can have a notched izod impact strength of at least 50 J/m, or at least 60 J/m, or at least 70 J/m, or at least 80 J/m, or at least 90 J/m, or at least 100 J/m, or at least 125 J/m, or at least 150 J/m, or at least 175 J/m, or at least 200 J/m, or at least 300 J/m, or at least 400 J/m, or at least 500 J/m, as measured according to ASTM D256 using a 3.2 mm thick bar that has been subjected to 50% relative humidity for 48 hours at 23° C. In certain embodiments, the polymer-based resin has a notched izod impact strength of at least 600 J/m, or at least 700 J/m, or at least 800 J/m, or at least 900 J/m, or at least 1000 J/m, as measured according to ASTM D256 using a 3.2 mm thick bar that has been subjected to 50% relative humidity for 48 hours at 23° C.

In certain embodiments, the copolyester composition, e.g., a PCTG copolyester blend with PC, has a ΔE value of less than 25, or less than 20, or less than 15, or less than 14, or less than 13, or less than 12, or less than 11, or less than 10, or less than 9, or less than 8, or less than 7, or less than 6, or less than 5, using a 3.2 mm plaque after injection molding with a barrel temperature of 260° C. and a mold temperature of 50° C., wherein ΔE is determined by the following equation: ((L*−100)²+(a*−0)²+(b*−0)²)^{1/2}, where the L*, a*, and b* color components were measured according to ASTM E1348. In certain embodiments, the polymer-based resin has a ΔE value in the range from 2 to 25, or from 2 to 20, or from 2 to 15, or from 2 to 14, or from 2 to 13, or from 2 to 12, or from 2 to 11, or from 2 to 10, or from 2 to 9, or from 2 to 8, or from 2 to 7, or from 2 to 6, or from 2 to 5, using a 3.2 mm plaque after injection molding with a barrel temperature of 250-280° C. and a mold temperature of 50° C., wherein ΔE is determined by the following equation: ((L*−100)²+(a*−0)²+(b*−0)²)^{1/2}, where the L*, a*, and b* color components were measured according to ASTM E1348.

In embodiments of the invention, the polymer-based resin has an L* color of at least 85, or at least 86, or at least 87, or at least 88, or at least 89, or at least 90, or at least 91, or at least 92, or at least 93, or at least 94, or at least 95, measured according to ASTM E1348 using a 3.2 mm plaque after injection molding with a barrel temperature of 260° C. and a mold temperature of 50° C. In certain embodiments, the polymer-based resin has an L* color in the range from 85 to 98, or from 85 to 97, or from 85 to 96, or from 85 to 95, measured according to ASTM E1348 using a 3.2 mm plaque after injection molding with a barrel temperature of 260° C. and a mold temperature of 50° C.

In embodiments of the invention, the polymer-based resin has a b* value is less than 15, or less than 12, or less than 10, or less than 9, or less than 8, or less than 7, or less than 6, or less than 5, or less than 4, measured according to ASTM E1348 using a 3.2 mm plaque after injection molding with a barrel temperature of 260° C. and a mold temperature of 50° C. In certain embodiments, the polymer-based resin has a b* color in the range from 0 to 15, or from 0 to 10, or from 0 to 8, or from 0 to 5, measured according to ASTM E1348 using a 3.2 mm plaque after injection molding with a barrel temperature of 260° C. and a mold temperature of 50° C.

In certain applications, shaped articles can be provided that are not continuously extruded films that are infinite (or continuous) in one direction and fixed in width and thickness in the other two directions, as would be the case in a rolled film. In certain embodiments, a film or sheet can be converted into a shaped article, e.g., by thermoforming into a three-dimensional object, such as a cup or bowl. In embodiments of the invention, the shaped article is not a film or is not a sheet. In embodiments of the invention, the shaped articles can be chosen from injection molded articles, extrusion molded articles, rotational molded articles, compression molded articles, blow molded articles, injection blow molded articles, injection stretch blow molded articles, extrusion blow molded articles, sheet or film extrusion articles, profile extrusion articles, gas assist molding articles, structural foam molded articles, or thermoformed articles.

Shaped articles made from the polyester compositions can be shaped via molding or extruding for use in vapor delivery applications. The shaped article can be chosen from transparent articles, see-through articles, thin-walled articles, technical articles (e.g., articles having a complex design), articles having high design specifications, intricate design articles, containers, food contact articles, household articles, general consumer products, packaging articles, medical articles, or components thereof, where the article is configured to receive a vapor deliver chemical containing composition. The polyester composition can be primary molded into forms such as pellets, plates, or parisons, and can then be secondary molded into articles, e.g., conduits, tubes, thin-wall vessels, or thick-wall vessels, configured to receive a vapor delivery chemical containing composition (as described herein).

The methods of forming the polyester compositions into films, molded articles, and sheeting can be according to methods known in the art. In embodiments, the polyester composition can be over molded onto itself or a different polyester composition and retain an interface bond (or weld line) strength that will not separate (or delaminate) when an article (having such an over mold interface) is used for its intended purpose. In embodiments, transparent polyesters and translucent (or opaque) polyesters can be over molded onto the other. In embodiments, the different polyesters all fall with one or more embodiments of the invention (as discussed herein).

In one aspect, an article is provided that comprises a molded component configured to receive a vapor deliver chemical containing composition, where the molded component is formed of a plastic composition comprising a copolyester composition and having a HDT of at least 85° C. In certain embodiments, the plastic composition has a HDT of at least 85° C. or at least 90° C.

In embodiments, the vapor delivery chemical containing composition comprises a vapor delivery chemical (VDC) that is present in an amount of at least 0.1, or at least 0.2, or at least 0.5, or at least 1, or at least 2, or at least 3, or at least 5, or at least 10, or at least 15, or at least 20, or at least 25 wt %, based on the total weight of the vapor delivery chemical containing composition.

Depending on the intended application, the vapor delivery chemical containing composition can contain one or more alcohols and/or polyols in an amount (total of all alcohols and/or polyols) of at least 1 wt %, or at least 5 wt %, or at least 10 wt %, or at least 15 wt % or at least 20 wt %, or at least 25 wt %, or at least 30 wt %, or at least 35 wt %, or at least 40 wt % or at least 45 wt %, or at least 50 wt %. In embodiments, the vapor delivery chemical containing composition contains one or more alcohols and/or polyols in an amount (total of all alcohols and/or polyols) of at least 55 wt %, or at least 60 wt %, or at least 65 wt %, or at least 70 wt %, or at least 75 wt %, or at least 80 wt %, or at least 85 wt %, or at least 90 wt %. In embodiments, the vapor delivery chemical containing composition contains one or more polyols in an amount of at least 40 wt %, or at least 45 wt %, or at least 50 wt %, at least 55 wt %, or at least 60 wt %, or at least 65 wt %, or at least 70 wt %, or at least 75 wt %, or at least 80 wt %, or at least 85 wt %, or at least 90 wt %. In embodiments, the one or more polyols are chosen from glycerol, propylene glycol, or combinations thereof. In embodiments, the vapor delivery chemical containing composition contains one or more alcohols chosen from ethyl alcohol, isoamyl alcohol, levomenthol, or combinations thereof.

Depending on the intended application, the vapor delivery chemical containing composition can also contain an oil or oil derivative in an amount of at least 0.01 wt %, or at least 0.02 wt %, or at least 0.05 wt %, or at least 0.1 wt %, or at least 0.2 wt %, or at least 0.5 wt %, or at least 1 wt %, or at least 5 wt %, or at least 10 wt %, or at least 15 wt % or at least 20 wt %, or at least 25 wt %. In embodiments, the oil is plant-based oil. The oil derivative can be derived from the oil, e.g., extracted from the oil. The definition of plants is not to be limited and can include any type or classification of plants, including vascular, non-vascular, seed bearing, spore bearing, angiosperms, and gymnosperms. Plants can include small plants, bushes, or trees. In embodiments, the plant-based oil can be synthesized or made without the oil actually being derived from plants, as long as the oil is of a type that can be found in or obtained from plants. In embodiments, the oil is a terpene containing oil or the oil derivative is a terpenoid, e.g., levomenthol. Other plant-based oil derivatives can include aldehyde, ester, acid, di- or tri-glyceride compounds.

In embodiments, the plant-based oil is a type found primarily in the leaves or flowers of a plant. In embodiments, the plant-based oil is a type found primarily in the seeds or fruit of a plant. In embodiments, the vapor deliver chemical containing composition can include a combination (e.g., mixture or blend) of different plant-based oils. In embodiments, the plant-based oil is a botanical oil. Botanical oil means an oil of a type obtained from plants that are fatty, dense and non-volatile. In embodiments, the botanical oil is extracted from the root, stem/bark, leaves, flowers, seeds or fruit of a plant, tree or shrub.

In embodiments, the botanical oil is cold pressed or extracted by heat. Examples of botanical oils can include rosehip oil (Rosa canina), evening primrose oil (Oenothera biennis), almond oil (Prunus amygdalus dulcis), calendula oil (Calendula officinalis), MCT oil, olive oil, canola oil, corn oil, vegetable oil, cotton seed oil, safflower oil, sunflower seed oil, soapbark tree oil; and extracts, isolates, or derivatives of the foregoing; and combinations of any of the foregoing.

In embodiments, the plant-based oil is an essential oil. Essential oil means a concentrated and volatile substance extracted from plants chosen from aromatic herbs or aromatic plants, where essential refers to an oil that carries a distinctive scent (or essence) of such a plant. Examples of essential oils can include agar oil or oodh, aiwain oil, angelica root oil, anise oil, asafetida oil, balsam of peru, basil oil, bay oil, bergamot oil, black pepper oil, buchu oil, birch oil, camphor oil, cannabis flower essential oil, calamodin oil or calamansi essential oil, caraway seed oil, cardamom seed oil, carrot seed oil, cedar oil, chamomile oil, calamus oil, cinnamon oil, cistus ladanifer, citron oil, citronella oil, clary sage oil, coconut oil, clove oil, coffee oil, coriander oil, costmary oil, costus root oil, cranberry seed oil, cubeb oil, cumin seed oil or black seed oil, cypress oil, cypriol oil, curry leaf oil, davana oil, dill oil, elecampane oil, elemi oil, eucalyptus oil, fennel seed oil, fenugreek oil, fir oil, frankincense oil, galangal oil, galbanum oil, garlic oil, geranium oil, ginger oil, goldenrod oil, grapefruit oil, henna oil, helichrysum oil, hickory nut oil, horseradish oil, hyssop, Idaho-grown tansy, jasmine oil, juniper berry oil, Laurus nobilis, lavender oil, ledum oil, lemon oil, lemongrass oil, lime oil, listea cubeba oil, linalool oil, mandarin oil, marjoram oil, melissa oil or lemon balm, Mentha arvensis oil or mint oil, moringa oil, mountain savory oil, mugwort oil, mustard oil, myrrh oil, myrtle oil, neem oil, neroli oil, nutmeg oil, orange oil, oregano oil, orris oil, palo santo oil, parsley oil, patchouli oil, perilla essential oil, pennyroyal oil, peppermint oil, petitgrain oil, pine oil, ravensara oil, red cedar oil, romain chamomile oil, rose oil, rosehip oil, rosemary oil, rosewood oil, sage oil, sandalwood oil, sassafras oil, savory oil, Schisandra oil, spearmint oil, spikenard oil, spruce oil, star anise oil, tangerine oil, tarragon oil, tea tree oil, thyme oil, tsuga oil, turmeric oil, warionia oil, vetiver oil, western red cedar oil, wintergreen oil, yarrow oil, ylang-ylang oil; and extracts, isolates, or derivatives of the foregoing; and combinations of any of the foregoing. In embodiments, the extract, isolate or derivative of the essential oil comprises a terpene or a flavonoid. In embodiments, the terpene is chosen from d-limonene, geraniol, b-pinene, myrcene, terpinolene, or mixtures thereof.

In embodiments, the plant-based oil can be a combination of one or more botanical oils and one or more essential oils. In embodiments, the vapor deliver chemical containing composition comprises plant-based oils chosen from a botanical oil, an essential oil, or combinations of botanical and essential oils. Examples of plant-based oils can include eucalyptus oil, lavender oil, neroli oil, Solanaceae (nightshade) family (e.g., Nicotiana tabacum or N. rustica species) plant oil, cannabis oil, hemp oil, cannabidiol oil, peppermint oil, sweet orange oil, tea tree oil, lemon oil, lime oil, orange oil; and extracts, isolates, or derivatives of the foregoing oils and/or their plant source; and combinations of any of the foregoing.

In embodiments, in combination with one or more oils (as discussed herein) or in the absence of such oils, the vapor deliver chemical containing composition can comprise one or more additives chosen from solvents, dispersants, stabilizers, emulsifiers, carriers, solvents, actives. In embodiments, the additive(s) can be chosen from glycols, e.g., propylene glycol, glycerin, e.g., plant glycerin, polysorbates, plant-based alkaloids, e.g., nicotine, or combinations thereof.

In embodiments, the vapor delivery chemical containing composition comprises nicotine, e.g., is an E-cig liquid formulation. In embodiments, in addition to nicotine, such compositions can contain significant amounts, e.g., in excess of 25 wt %, or 30 wt % of the composition, of one or more glycols and/or polyols.

In embodiments, the vapor delivery chemical containing composition can contain one or more of the following: glycerol in an amount from 0 to 99 wt %, propylene glycol in an amount from 0 to 99 wt %, ethyl alcohol in an amount from 0 to 10 wt %, nicotine in an amount from 0 to 5 wt %, acetic acid isobutyl ester in an amount from 0 to 5 wt %, isoamyl acetate in an amount from 0 to 5 wt %, carvone in an amount from 0 to 5 wt %, triacetin in an amount from 0 to 5 wt %, diacetin in an amount from 0 to 5 wt %, levomenthol in an amount from 0 to 5 wt %, isoamyl alcohol in an amount from 0 to 2 wt %, 2-methylbutyric acid in an amount from 0 to 2 wt %, and vanillin in an amount from 0 to 2 wt %.

In embodiments, the vapor delivery chemical containing composition comprises one or more of the following: glycerol in an amount from 30 to 50 wt %, or 35 to 45 wt %, or 40 to 45 wt %; propylene glycol in an amount from 40 to 70 wt %, or 45 to 65 wt %, or 50 to 60 wt %; and nicotine in an amount from 0.1 to 2 wt %, or 0.2 to 1.5 wt %, or 0.5 to 1.5 wt %. In embodiments, the vapor delivery chemical containing composition further comprises one or more of the following: ethyl alcohol in an amount from 0.1 to 10 wt %, or 0.2 to 5 wt %, or 0.5 to 3 wt %; isoamyl alcohol in an amount from 0.01 to 1 wt %, or 0.02 to 0.5 wt %, or 0.05 to 0.15 wt %; 2-methylbutyric acid in an amount from 0.01 to 1 wt %, or 0.02 to 0.5 wt %, or 0.02 to 0.1 wt %; and vanillin in an amount from 0.01 to 1 wt %, or 0.02 to 0.5 wt %, or 0.02 to 0.1 wt %.

In one embodiment, the vapor delivery chemical containing composition is an E-cig liquid formulation shown in Table 1 below.

TABLE 1
E-cig liquid formulation

| | Concentration and tolerance |
|---|---|
| Component | g/100 g |
| Propylene glycol | 38 ± 2 |
| Glycerol | 48 ± 2 |
| Water | 2.0 ± 0.1 |
| Ethyl alcohol | 1.00 ± 0.05 |
| Nicotine | 1.00 ± 0.05 |
| Essence (See Table 2) | 10.0 ± 0.5 |

TABLE 2
Essence formulation in E-cig liquid

| | Concentration and tolerance |
|---|---|
| Component | g/100 g |
| Vanillin | 1.00 ± 0.05 |
| isoamyl alcohol | 2.0 ± 0.1 |
| 2-methyl butyric acid | 1.00 ± 0.05 |
| Propylene glycol | 6.0 ± 0.5 |

### Polyester Properties

Properties disclosed herein requiring a test method can be determined as follows:

### Test Methods

Properties disclosed can be determined according to the test methods described herein. Samples were evaluated using standard ASTM test methods (in Table 3) with any special conditions noted below.

TABLE 3
Test Methods

| PROPERTY | COMMENTS |
|---|---|
| Color, b* (ASTM E1348) | Using 3.2 mm thick plaques. Measured using |
| | Hunter Lab Ultrascan Spectra Colorimeter |
| Color, a* (ASTM E1348) | Using 3.2 mm thick plaques. Measured using |
| | Hunter Lab Ultrascan Spectra Colorimeter |
| Color, L* (ASTM E1348) | Using 3.2 mm thick plaques. Measured using |
| | Hunter Lab Ultrascan Spectra Colorimeter |
| Haze (ASTM D1003) | Using 3.2 mm plaques |
| Transmission (ASTM D1003) | Using 3.2 mm plaques |
| Izod Notched Impact at 23° C. (ASTM D256) | Using a 3.2 mm thick bar that has been |
| | subjected to 50% relative humidity at 23° C. for |
| | 48 hours. |
| Dry Tg (ASTM D3418) | DSC at 20° C./min. Using pellet. |
| Tm (ASTM D2117) | DSC at 20° C./min. Using pellet |
| HDT (ASTM D648) | Using a 125 mm × 12.7 mm × 3.2 mm bar |
| | subjected to 50% Relative humidity for 40 |
| | hours at 23° C. |
| MFR (ASTM D1238) | Using pellet, 2.16 kg loading |
| Tensile Modulus, Tensile strength @ Yield, Tensile | Using a 165 mm × 13 mm × 3.2 mm bar |
| strength @break (ASTM D638) | subjected to 50% Relative humidity for 40 |
| | hours at 23° C. |
| Flex Modulus (ASTM D790) | Using a 125 mm × 12.7 mm × 3.2 mm bar |
| | subjected to 50% Relative humidity for 40 |
| | hours at 23° C. |

The inherent viscosity of the polyesters was determined in 60/40 (wt/wt) phenol/tetrachloroethane at a concentration of 0.5 g/100 ml at 25° C. (according to ASTM D4603).

The glycol content was determined by proton nuclear magnetic resonance (NMR) spectroscopy. All NMR spectra were recorded on a JEOL Eclipse Plus 600 MHz nuclear magnetic resonance spectrometer using either chloroform-trifluoroacetic acid (70-30 volume/volume). Peak assignments for 2,2,4,4-tetramethyl-1,3-cyclobutanediol resonances were made by comparison to model mono- and dibenzoate esters of 2,2,4,4-tetramethyl-1,3-cyclobutanediol. These model compounds closely approximate the resonance positions found in the polymers.

The crystallization half-time, t_{1/2}, was determined by measuring the light transmission of a sample via a laser and photo detector as a function of time on a temperature controlled hot stage. This measurement was done by exposing the polymers to a temperature, Tₘₐₓ, and then cooling it to the desired temperature. The sample was then held at the desired temperature by a hot stage while transmission measurements were made as a function of time. Initially, the sample was visually clear with high light transmission and became opaque as the sample crystallized. The crystallization half-time was recorded as the time at which the light transmission was halfway between the initial transmission and the final transmission. Tₘₐₓ is defined as the temperature required to melt the crystalline domains of the sample (if crystalline domains are present). The Tₘₐₓ reported in the examples below represents the temperature at which each sample was heated to condition the sample prior to crystallization half time measurement. The Tₘₐₓ temperature is dependent on composition and is typically different for each polyester. For example, PCT may need to be heated to some temperature greater than 290° C. to melt the crystalline domains.

Differential scanning calorimetry (DSC) was performed using TA Instruments Model 2920 with a liquid nitrogen cooling accessory. The sample weight, in the range of 8 to 12 mg, was measured and recorded. Samples were first heated (1^{st} heating scan) from 0 to 320° C. at 20° C./min, followed by cooling to 0° C. at 20° C./min (cooling scan), and then heated again from 0 to 320° C. at 20° C. min. Various thermal parameters were measured and recorded. ΔH_{cc} (cal/g) is the heat of crystallization measured from the cooling scan. T_{cc} is the crystallization peak temperature on the cooling scan. T_{g} is the glass transition temperature measured from 2^{nd} heating scan. Tₘ is the melting point measured during the 2^{nd} heating scan. ΔH_{ch1} (cal/g) is the heat of crystallization measured during the 1^{st} heating scan. ΔHₘ₁ (cal/g) is the heat of melting measured during the 1^{st} heating scan.

The percent crystallinity formed during cooling is calculated by equation (1), assuming a specific heat of fusion of 29 cal/g (based on unmodified PCT).

$${X}_{c} = \frac{( Δ {H}_{c c} )}{29} \times 100 \qquad ( 1 )$$

The peak temperature in the crystallization exotherm (T_{cc}) occurs at 227° C. for unmodified PCT.

As used herein, the abbreviation “wt” means “weight”. Unless indicated otherwise, parts are parts by weight, temperature is in degrees C. or is at room temperature, and pressure is at or near atmospheric.

### EXAMPLES

Copolyester materials/compositions that were tested were as follows:
1) Blend 1 (B1): PC 2458/DN011 (70/30 wt %)
2) Blend 2 (B2): PC 2458/DN011 (50/50 wt %)
3) Blend 3 (B3): PC 2458/DN011 (30/70 wt %)
4) Blend 4 (B4): PC 2458/JN200 (70/30 wt %)
5) Blend 5 (B5): PC 2458/JN200 (50/50 wt %)
6) Blend 6 (B6): PC 2458/JN200 (30/70 wt %)
7) Comp 1 (C1): TX1501
8) Comp 2 (C2): TX1501/DN011 (70/30 wt %)
9) Comp 3 (C3): TX1501/DN011 (50/50 wt %)
10) Comp 4 (C4): TX1501/DN011 (30/70 wt %)
11) Comp 5 (C5): TX1501/JN200 (70/30 wt %)
12) Comp 6 (C6): TX1000/PC2458 (70/30 wt %)
13) Comp 7 (C7): TX1000/PC2458 (50/50 wt %)
   Polymeric materials/compositions that were used to make blends or used for tests were as follows:
1) PC 2458 (MAKROLON polycarbonate PC2458 from Covestro)
2) DN011 (Eastar Copolyester DN011 from Eastman)
3) JN200 (SKYGREEN® JN200 PCTG from SK Chemicals)
4) Copolyester TX1001 (from Eastman Chemical Company)
5) Copolyester TX1501 (from Eastman Chemical Company)
   Polymeric blends based on PC and PCTG were compared to blends containing a copolyester having a cyclobutane diol residue (“CBDO Polyester”).

### Example 1

### Blend Testing

Blends were made using a twin-screw extruder with screw diameter of 26 mm (Coperion ZSK 26 Mc18). The components were pre-blended (e.g., pellets were bag blended) and the pre-blend was fed to the extruder and compounded by extruding under a 260° C. melt temperature setting.

Certain physical properties were measured for the different blends. The results are listed below in Tables 4 and 5.

TABLE 4
CBDO and CBDO/PCTG or PC Blends

| Property | C1 | C2 | C3 | C4 | C5 | C6 | C7 |
|---|---|---|---|---|---|---|---|
| IV (dL/g) | 0.63 | 0.678 | 0.691 | 0.719 | 0.665 | 0.69 | 0.705 |
| Dry Tg (° C.) | 105 | 96.42 | 90.51 | 87.93 | 96.97 | 118.75 | 126.78 |
| HDT-.445 | 94 | 84.7 | 80 | 78.9 | 83 | 107.7 | 117.7 |
| MPa (° C.) | | | | | | | |

TABLE 5
PC/PCTG Blends

| Property | B1 | B2 | B3 | B4 | B5 | B6 |
|---|---|---|---|---|---|---|
| IV (dL/g) | 0.712 | 0.713 | 0.714 | 0.697 | 0.712 | — |
| Dry Tg (° C.) | 125 | 111 | 99 | 122 | 110 | 98 |
| HDT-.445 | 116 | 103 | 91 | 115 | 101 | 90 |
| MPa (° C.) | | | | | | |

The blends were subjected to testing for mechanical properties. The results are listed below in Tables 6 and 7.

TABLE 6
CBDO and CBDO/PCTG or PC Blends

| Property | C1 | C2 | C3 | C4 | C5 | C6 | C7 |
|---|---|---|---|---|---|---|---|
| Tensile Strength (MPa) | 50.8 | 48.5 | 48.1 | 48.9 | 47.6 | 56.2 | 62.0 |
| Tensile Strength @ | 50.8 | 48.5 | 47.0 | 46.6 | 47.2 | 56.2 | 46.6 |
| break (MPa) | | | | | | | |
| Tensile Elong. (%) | 162 | 153 | 169 | 171 | 151 | 140 | 171 |
| Tensile Mod. (MPa) | 1680 | 1700 | 1753 | 1800 | 1730 | 1840 | 1800 |
| Flex. Mod. (MPa) | 1575 | — | — | — | — | 2038 | — |

TABLE 7
PC/PCTG Blends

| Property | B1 | B2 | B3 | B4 | B5 | B6 |
|---|---|---|---|---|---|---|
| Tensile Strength (MPa) | 64.1 | 60.6 | 56.2 | 65.4 | 62.3 | 56.3 |
| Tensile Strength @ | 61 | 60.6 | 55 | 64.4 | 62.3 | 51.5 |
| break (MPa) | | | | | | |
| Tensile Elong. (%) | 125 | 142 | 150 | 134 | 155 | 153 |
| Tensile Mod. (MPa) | 2360 | 2210 | 2050 | 2350 | 2260 | 2090 |
| Flex. Mod. (MPa) | 2499 | 2372 | 2220 | 2540 | 2451 | 2325 |

Certain blends were subjected to testing for optical properties. Transmission and haze were measured by haze meter according to ASTM D1003. The results are listed below in Table 8.

TABLE 8
PC/PCTG and CBDO/PCTG or PC Blends

| Property | B6 | C2 | C3 | C5 | C6 |
|---|---|---|---|---|---|
| Trans. (%) | 89.8 | 89.2 | 89.2 | 88.5 | 89.4 |
| Haze (%) | 0.52 | 2.64 | 4.37 | 4.32 | 0.95 |
| b* | 1.17 | — | — | — | 0.63 |

Chemical compatibility of the blends was tested by exposing a tensile bar to a specified chemical and determining the change in tensile elongation. Testing was conducted as follows:

### ESCR-Property Retention in Tensile Elongation

Testing was conducted using injection molded tensile bars with length, width, and thickness of 211 mm (8.3″), 13 mm (0.5″), and 3.2 mm (0.125″), respectively. Bars were conditioned at 23° C./50% RH for a minimum of 72 hr. Bars were clamped into a constant strain fixture or a 3-point bend fixture at 1.5% strain and exposed to test substance using a cotton pad saturated with the test substance, where the pad was placed on the top surface of the bar. After the test substances were applied to the bars on the side without ejector pin marks, the strain fixtures with bars attached were sealed in polyethylene bags for 24 hours at nominal temperature of 23° C., after which the bars were wiped clean and removed from the strain fixture.

After exposure, the bars were tested at 23° C. for tensile elongation. The test apparatus was a Instron universal tester, and the test method follow ASTM D638. Control bars were tensile tested in addition to bars that were exposed to the test substances. The comparison of results between the controls and the chemically exposed bars was used to calculate percent retention of original tensile elongation. The test was repeated three times and the results are an average of the three tests.

The test substances used in the tensile elongation retention testing were propylene glycol, glycerol, polyethylene glycol-MW 400 (PEG 400), and several commercial E-cig oils listed in Table 9 below.

TABLE 9
Commercial E-cig oils

| E-cig Oil | Source/Product |
|---|---|
| EC-1 | Doctor-CIT Lemon |
| EC-2 | Chazu-blue ice cola |
| EC-3 | HYAKKI- black beer |
| EC-4 | Relx-passion fruit |
| EC-5 | Relx-mint |
| EC-6 | Relx-watermelon |
| EC-7 | Relx-Green bean |

A semi-quantitative analysis (by GC-mass spectrometry) revealed that all the E-cig oils in Table 9 contained significant levels of both glycerol and propylene glycol, where the combined amount accounted for a majority of each oil (i.e., it is believed the total combined amount of glycerol and propylene glycol was at least 50 wt % of each of these oils). The results for the blends for exposure to chemicals/substances are shown below in Tables 10 and 11.

TABLE 10
Percent Retention of Tensile Elongation After Exposure
for neat polymers and CBDO/PCTG or PC blends

| Test Substance | TX1501 | PC2458 | C2 | C3 | C4 | C5 | C6 | C7 |
|---|---|---|---|---|---|---|---|---|
| EC-1 | 4% | 100% | 103% | 99% | 39% | 100% | 5% | 8% |
| EC-2 | 25% | 41% | 87% | 75% | 96% | 96% | 4% | — |
| EC-3 | 2% | 2% | 95% | 90% | 96% | 89% | 81% | 31% |
| EC-4 | 3% | 5% | 54% | 89% | 96% | 93% | 75% | — |
| EC-5 | 19% | 7% | 29% | 45% | 95% | 58% | 4% | — |
| EC-6 | 74% | 2% | 79% | 85% | 91% | 78% | 5% | — |
| EC-7 | 75% | 88% | 91% | 91% | 89% | 93% | 61% | — |

TABLE 11
Percent Retention of Tensile Elongation
After Exposure for PC/PCTG blends

| Test Substance | TX1501 | PC2458 | B1 | B2 | B3 | B4 | B5 | B6 |
|---|---|---|---|---|---|---|---|---|
| EC-1 | 4% | 100% | 2% | 4% | 82% | 3% | 2% | 97% |
| EC-2 | 25% | 41% | 2% | 4% | 82% | 2% | 3% | 97% |
| EC-3 | 2% | 2% | 2% | 75% | 95% | 3% | 31% | 94% |
| EC-4 | 3% | 5% | 1% | 2% | 41% | 1% | 2% | 82% |
| EC-5 | 19% | 7% | 1% | 2% | 86% | 1% | 2% | 43% |
| EC-6 | 74% | 2% | 1% | 3% | 22% | 1% | 1% | 8% |
| EC-7 | 75% | 88% | 2% | 5% | 88% | 1% | 2% | 82% |

A review of Tables 10 and 11 reveals that blends B3 and B6 performed well for the EC chemicals tested and outperformed comparative blends C6 and C7 (that had the higher Tg values) for the EC-1 and EC-3 test substances.

1. An article comprising a molded component configured to receive a vapor delivery chemical containing composition (“VDC”), said molded component formed from a copolyester composition comprising a polymeric component that comprises a blend of different polymers, said blend comprising a first copolyester and a second polymer,
wherein the first copolyester comprises:
(a) a dicarboxylic acid component comprising:
i) 50 to 100 mole % of terephthalic acid or dimethyl terephthalate residues;
(b) a glycol component comprising:
i) 0 to 50 mole % of ethylene glycol residues; and
ii) 50 to 100 mole % of 1,4-cyclohexanedimethanol residues, wherein the total mole % of the dicarboxylic acid component is 100 mole %, and the total mole % of the glycol component is 100 mole %; and have an inherent viscosity of 0.50 to 1.1 dL/g as determined in 60/40 (wt/wt) phenol/tetrachloroethane at a concentration of 0.5 g/100 ml at 25° C.;
wherein the second polymer is a polycarbonate (PC);
wherein the copolyester composition has a heat deflection temperature (HDT) of 85° C. or greater, measured per ASTM D648 at 0.45 MPa load.

2. The article according to claim 1, wherein the copolyester composition has an HDT in the range from 85° C. to 120° C.

3. The article according to claim 2, wherein the copolyester composition has an inherent viscosity is 0.65 to 1.0 dL/g.

4. The article according to any of claim 1, wherein the glycol component comprises:
i) 33 to 43 mole % of ethylene glycol residues; and
ii) 57 to 67 mole % of 1,4-cyclohexanedimethanol residues.

5. The article according to claim 4, wherein the dicarboxylic acid component of the first copolyester comprises 100 mole % terephthalic acid or dimethyl terephthalate residues.

6. The article according to claim 1, wherein the first polymer is present in an amount greater than 50 wt % and the second polymer is present in an amount less than 50 wt %, based on the total weight of the copolyester composition.

7. The article according to claim 1, wherein the copolyester composition has a haze value less than 10, measured according to ASTM D1003 using a 3.2 mm plaque after injection molding at a barrel set point of 250 to 270° C. and a mold temperature of 50° C.

8. The article according to claim 7, wherein the copolyester composition has a has a b* value less than 5, measured according to ASTM E1348 using a 3.2 mm plaque after injection molding with a barrel temperature of 260° C. and a mold temperature of 50° C.

9. The article according to claim 1, comprising a VDC in contact with a surface of said molded component.

10. The article according to claim 9, wherein the VDC is in the form of a pre-vapor formulation and/or a vapor.

11. The article according to claim 1, wherein the molded component comprises a container configured to contain a VDC and selectively release the VDC.

12. The article according to claim 11, wherein the molded component comprises a container configured to contain a VDC that is in a pre-vapor formulation form.

13. The article according to claim 10, wherein the molded component comprises a conduit configured to convey a VDC.

14. The article according to claim 13, wherein the molded component comprises a conduit configured to convey a VDC that is in a vapor form.

15. The article according to claim 14, wherein the article comprises one or more molded components that comprise a container configured to contain a VDC and a conduit configured to convey a VDC, wherein the container and conduit are in fluid communication.

16. The article according to claim 15, wherein the article is a vapor delivery device configured to deliver a vapor comprising a VDC.

17. The article according to claim 16, wherein the vapor delivery device is chosen from a vaporizer, nebulizer, humidifier, air freshener, or hand-held vapor delivery device.

18. The article according to claim 1, wherein the molded component can be chosen from injection molded articles, extrusion molded articles, rotational molded articles, compression molded articles, blow molded articles, injection blow molded articles, injection stretch blow molded articles, extrusion blow molded articles, sheet or film extrusion articles, profile extrusion articles, gas assist molding articles, structural foam molded articles, or thermoformed articles.

19. The article according to claim 1, wherein the dicarboxylic acid component of the first copolyester comprises monomer residues having at least 25 mole % recycle content.
